(19) United States
(12) Patent Application Publication
(10) Pub. No.: US 2026/0083485 A1
(43) Pub. Date: Mar. 26, 2026

(54) TORQUE TRANSFER MECHANISMS

(71) Applicant: NuVasive Specialized Orthopedics, INC., San Diego, CA (US)

(72) Inventors: Nathan Meyer, Vista, CA (US); Jorge Lopez Camacho, Oxnard, CA (US); Woong Kim, Fresno, CA (US); Robert Patterson, Aliso Viejo, CA (US); Dalton Jennings, Aliso Viejo, CA (US); Gabriel Buenviaje, Laguna Hills, CA (US); Shanbao Cheng, Rancho Santa Margarita, CA (US)

(21) Appl. No.: 19/112,768

(22) PCT Filed: Sep. 8, 2023
(86) PCT No.: PCT/US23/73708

§ 371 Date: Mar. 18, 2025

Related U.S. Application Data
(60) Provisional application No. 63/375,481, filed on Sep. 13, 2022.

(51) Int. Cl. A61B 17/70; A61B 17/00

(52) U.S. Cl. CPC A61B 17/7082; A61B 2017/00876

(57) ABSTRACT

Implantable medical devices are disclosed herein, having a biocompatible housing; a driver of rotational motion; a receiver of rotational motion; and an interface configured to transfer torque from the driver to the receiver. In various embodiments, the interface comprises magnetic, mechanical, or magnetic and mechanical elements for transferring torque.

### CROSS REFERENCE TO RELATED APPLICATIONS

The present patent application claims priority to US provisional Ser. No. 63/375,481, filed 13 Sep. 2022.

### BACKGROUND OF THE INVENTION

The invention relates generally to methods and interfaces for transferring torque from a driver of rotational motion to a receiver of rotational motion. More particularly, the invention relates to implantable medical devices including a driver of rotational motion, a receiver of rotational motion, and an interface disposed therebetween for transferring torque from the driver to the receiver.

Various implant systems, including spinal distraction and compression systems, intramedullary and extramedullary distraction and compression systems, and the like, function in part by transferring rotational movement from a driver to a receiver in order for the implant to achieve the desired distraction or compression.

Torque transfer interfaces for use in implantable medical devices exist in the prior art. For example, U.S. Pat. No. 9,848,914 (filed 2014 Jul. 15) and U.S. Pat. No. 9,421,046 (filed 2014 Aug. 04), which are both incorporated herein by reference in their entirety for any and all purposes, describe torque transfer interfaces that include the use of pins and aperture arrangements to facilitate the transfer of torque from a driving element to a lead screw as the receiving element in an implant. However, torque transfer components with additional capabilities and features may be desirable. For example, torque transfer interfaces that are capable of side loading assembly rather than axial assembly within the implant may provide ease of assembly. Interfaces that allow axial translation of one or both of the mechanisms while maintaining torque transfer may also be desirable. Still further, interfaces that allow for, or are adaptable to non-concentric shaft arrangements, that facilitate torque transfer without direct contact between components, and/or that provide float between components may be desirable. Some degree of float may permit components to connect in spite of any minor imperfections in the pieces themselves.

Accordingly, mechanical and magnetic torque transfer methods and components are provided herein, to address these and other challenges.

### BRIEF DESCRIPTION OF THE INVENTION

A first aspect of the disclosure provides an implant comprising a biocompatible housing; a driver; a receiver; and an interface configured to transfer torque from the driver to the receiver. The interface comprises a first magnetic member rotationally fixed to the driver, wherein the first magnetic member comprises a dipole magnet, and a rotation of the driver is configured to cause a rotation of the first magnetic member. A second magnetic member is rotationally fixed to the receiver, wherein a rotation of the second magnetic member is configured to cause a rotation of the receiver, wherein the first magnetic member is configured to attract the second magnetic member, and the second magnetic member is configured to rotate in response to a rotation of the first magnetic member.

In some embodiments, the second magnetic member is configured to rotate more slowly than the first magnetic member. For example, the rotation of the second magnetic member may lag behind the rotation of the first magnetic member.

In some embodiments, the second magnetic member comprises a dipole magnet.

In some embodiments, the first magnetic member is magnetically coupled with the second magnetic member.

In some embodiments, the first magnetic member comprises a disc-shaped dipole magnet, and the second magnetic member comprises a disc-shaped dipole magnet.

In some embodiments, the interface further comprises a component disposed between the first magnetic member and the second magnetic member, the component including a thrust bearing or a friction reducing component.

In some embodiments, the first magnetic member comprises a cross sectional shape configured to provide a complementary fit with a cross sectional shape of the second magnetic member.

In some embodiments, the second magnetic member is disposed at least partly within the first magnetic member in a male to female arrangement, and wherein the first magnetic member and the second magnetic member are substantially axially aligned.

In some embodiments, the first magnetic member further comprises a barrel or shape, and the second magnetic member further comprises a solid cylindrical shape, wherein the first magnetic member and the second magnetic member are substantially concentrically arranged.

In some embodiments, the second magnetic member comprises a magnetic material.

In some embodiments, the first magnetic member comprises a cross sectional shape configured to provide a complementary fit with a cross sectional shape of the second magnetic member.

In some embodiments, the second magnetic member is disposed at least partly within the first magnetic member in a male to female arrangement, and wherein the first magnetic member and the second magnetic member are substantially axially aligned.

In some embodiments, the first magnet member and the second magnetic member are rotationally locked relative to one another, and are axially free relative to one another.

In some embodiments, the first magnetic member further comprises a first engagement surface, and the second magnetic member comprises a second engagement surface, wherein the second magnetic member is configured to rotate in response to force exerted by the first engagement surface on the second engagement surface.

In some embodiments, the second magnetic member comprises a cross sectional geometry selected from: a semicircle, an irregular shape having at least one arc and one flat side, an irregular shape having at least one arc and two flat sides, an irregular shape having at least one arc and three flat sides, an irregular shape having at least one arc and four or more flat sides, a slotted circle, a cross or Phillips head, a hexalobular shape, and a keyed shape.

A second aspect of the disclosure provides an implant comprising a biocompatible housing; a driver; a receiver; and an interface configured to transfer torque from a driver to a receiver. The interface comprises a receptacle disposed on an end of the driver, the receptacle being configured to accommodate an end portion of the receiver therein. The receptacle includes a first receptacle groove and a second receptacle groove, each disposed on an annular inner side wall of the receptacle; and a first receiver groove and a second receiver groove, each disposed on a radially outer surface of the end portion of the receiver. When the end portion of the receiver is disposed within the receptacle, the first receptacle groove and the first receiver groove mate to form a first annular space, and the second receptacle groove and the second receiver groove mate to form a second annular space. A first toroidal spring is disposed within the first annular space, canted in a first direction; and a second toroidal spring disposed within the second annular space, canted in a second direction, where the second direction is opposite the first direction.

In some embodiments, each of the first toroidal spring and the second toroidal spring comprise a coil spring having a first end thereof welded to a second end thereof.

In some embodiments, the first toroidal spring has the same diameter as the second toroidal spring.

In some embodiments, the first toroidal spring has a first diameter, and the second toroidal spring has a second diameter that is different than the first diameter.

In some embodiments, the first end of the receiver comprises a shaft having a stepped diameter shaft, and the first annular space having a first diameter is disposed at a first step, and the second annular space having a second diameter is disposed at a second step.

In some embodiments, the end portion of the receiver further comprises a shaft and a collar disposed thereon, wherein one of the first annular space or the second annular space is disposed on the collar, and the other of the first annular space or the second annular space is disposed on the shaft itself.

These and other aspects, advantages and salient features of the invention will become apparent from the following detailed description, which, when taken in conjunction with the annexed drawings, where like parts are designated by like reference characters throughout the drawings, disclose embodiments of the invention.

### BRIEF DESCRIPTION OF THE DRAWINGS

FIG. 1 illustrates a perspective view of an implantable device in accordance with embodiments of the disclosure.

FIG. 2 illustrates a cross sectional view of an implantable device in accordance with embodiments of the disclosure.

FIG. 3 illustrates a cross sectional view of a portion of an implantable device in accordance with embodiments of the disclosure.

FIG. 4 illustrates a perspective view of a portion of an implantable device including the gear stages, in accordance with embodiments of the disclosure.

FIG. 5 illustrates a perspective view of a portion of an implantable device including the gear stages in accordance with embodiments of the disclosure.

FIG. 6 illustrates a perspective view of the portion of an implantable device of FIG. 4 with a ring gear disposed about the gears, in accordance with embodiments of the disclosure.

FIG. 7 shows a perspective view of a first magnetic torque transfer interface in accordance with an embodiment of the disclosure.

FIG. 8 shows an exploded perspective view of a second magnetic torque transfer interface in accordance with an embodiment of the disclosure.

FIG. 9 shows a perspective view of a third magnetic torque transfer interface in accordance with an embodiment of the disclosure.

FIG. 10A shows a cross sectional view of a toroidal spring torque transfer interface in accordance with an embodiment of the disclosure.

FIGS. 10B-10C show top-down cross sectional views of portions of a toroidal spring torque transfer interface in accordance with an embodiment of the disclosure.

FIG. 11A shows a perspective view of a mechanical torque transfer interface in accordance with an embodiment of the disclosure.

FIGS. 11B-11I show perspective views of portions of mechanical torque transfer interfaces in accordance with embodiments of the disclosure.

FIGS. 11J-11L show side, perspective, and end, views, respectively, of portions of a mechanical torque transfer interface in accordance with an embodiment of the disclosure.

FIGS. 11J-11L show side, perspective, and end, views, respectively, of portions of a mechanical torque transfer interface in accordance with an embodiment of the disclosure.

FIG. 11M shows a perspective view of portions of a mechanical torque transfer interface in accordance with an embodiment of the disclosure.

FIG. 11N shows a perspective view of portions of a mechanical torque transfer interface including a keyway in accordance with an embodiment of the disclosure.

FIG. 11O shows a perspective view of portions of a mechanical torque transfer interface including a pair of keyways in accordance with an embodiment of the disclosure.

FIG. 12 shows a perspective view of portions of a torque transfer interface including a pin and shank sleeve coupling in accordance with an embodiment of the disclosure.

FIGS. 13A-13B show a top-down sectional view and a side sectional view of portions of a mechanical torque transfer interface including a set screw in accordance with an embodiment of the disclosure.

FIGS. 14A-14B show perspective views of portions of a mechanical torque transfer interface including a T-slot in accordance with an embodiment of the disclosure.

FIGS. 15A-15B show a side views of portions of a mechanical torque transfer interface including a spline and retaining ring in accordance with an embodiment of the disclosure.

FIG. 16 shows an exploded perspective view of portions of a mechanical torque transfer interface including multiple pins in accordance with an embodiment of the disclosure.

FIGS. 17A-17B shows perspective views of portions of a mechanical torque transfer interface in accordance with an embodiment of the disclosure.

FIGS. 18A-18B show a side cross sectional view and a perspective view of a portion of a mechanical torque transfer interface in accordance with an embodiment of the disclosure.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION OF THE INVENTION

Various embodiments of the present invention provide interfaces for transferring torque from a driver to a receiver in an implantable medical device system, or “implant” as used herein. Such implants may also be known as bone transport devices or distraction devices, and may include spinal distraction and compression systems, intramedullary distraction and compression systems, extramedullary distraction and compression systems, and the like. Although certain embodiments of the invention are illustrated relative to an implant in the form of an intramedullary distraction device, it is understood that the teachings are equally applicable to other distraction and compression systems.

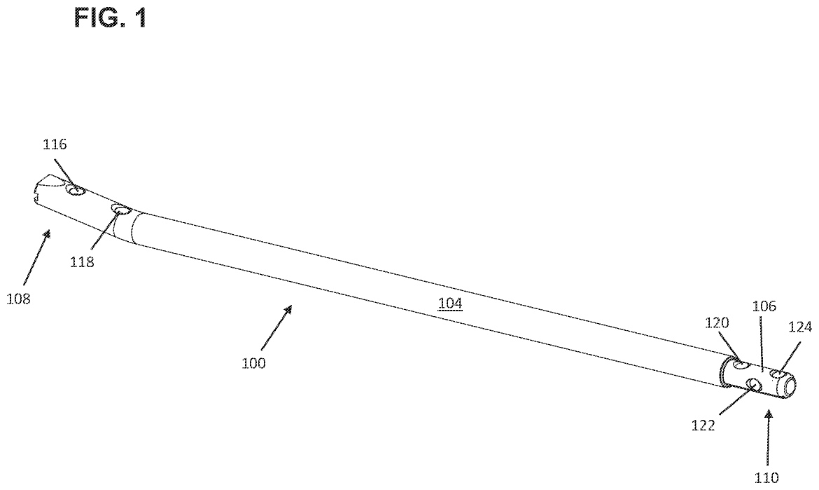

FIG. 1 illustrates an exemplary implant 100 having an adjustable portion 104 and a distraction rod 106. The implant 100 is configured to be affixed at its first end 108 to a first section of bone, and at its second end 110 to a second section of bone. A variety of different rod configurations may be used, each having a particular angulation and length, depending on the condition to be treated. Multiple configurations are contemplated for tibial and femoral use, in both antegrade and retrograde positions, as well as spinal distraction and compression use. In FIG. 1, the adjustable portion 104 is angled, while distraction rod 106 is straight. However, in other embodiments, the adjustable portion 104 may be straight through first end 108. Holes 116, 118, 120, 122, 124 are configured with specific diameters and orientations, in order to accommodate bone screws within each respective hole for securing the implant 100 to a section of bone. The implant 100 may be used to carry out distraction and compression procedures as is understood in the art and as described in, e.g., U.S. Pat. Nos. 9,848,914 and No. 9,421,046, which were previously incorporated herein by reference.

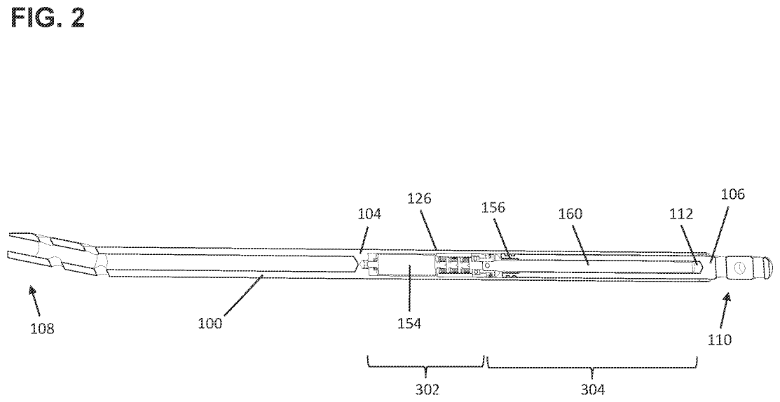

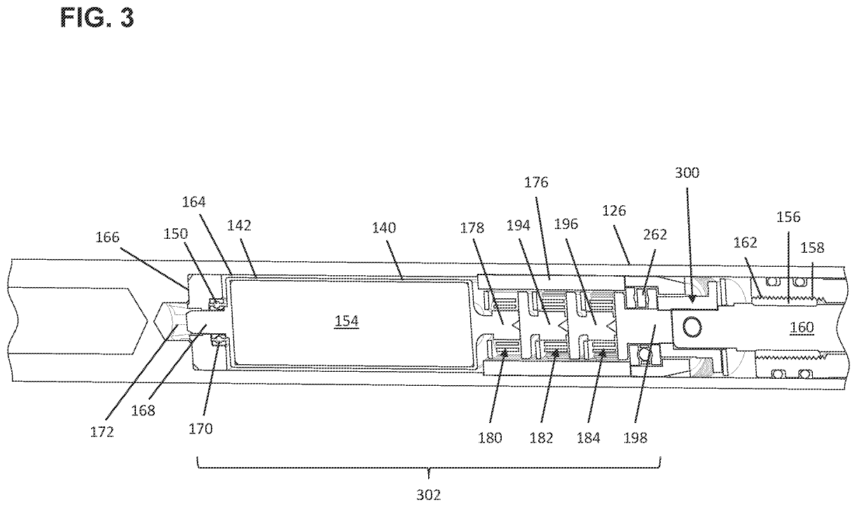

FIGS. 2-3 provide cross-sectional views illustrating the interaction between the distraction rod 106 with the adjustable portion 104. As best seen in FIG. 2, an end 110 of the distraction rod 106 includes an elongate recess 112 disposed therein, and open to an end disposed within the adjustable portion 104. The recess 112 is dimensioned to receive a lead screw 160. The lead screw 160 may be made from a high strength material such as, for example, titanium. As shown in the more detailed view in FIG. 3, at least a portion of the lead screw 160 includes external threads 162 that are configured to engage a nut 156 integrated into the recess 112. The nut 156 provides a threaded portion on the inner surface of the recess 112 of the distraction rod 106. The lead screw 160 may have, for example, 80 threads per inch, although more or fewer could be used. The nut 156 may include threads or a chamfered surface on the outer diameter in order to facilitate a secure attachment to the inner diameter of the recess 112 of the distraction rod 106. For example, the nut 156 may be bonded to the distraction rod 106 using an adhesive. This allows the distraction rod 106 to be fabricated from a single piece of stronger material. It also provides for clearance between the lead screw 160 and the internal diameter of the distraction rod 106. Alternatively, a threaded portion may be directly formed in the recess 112 without the aid of a separate nut 156.

The nut 156 includes internal threads 158 that engage with the outer threads 162 of the lead screw 160. In certain embodiments, the nut 156 may be made from aluminum-bronze #630. The use of dissimilar metals having different hardness values, e.g., titanium for the lead screw 160 and aluminum-bronze for the nut 156, may result in less gall/bind between the lead screw 160 and the nut 156. This further enables to the lead screw 160 and the nut 156 to operate with reduced friction. Optionally, various wet or dry lubricants may be used to reduce friction between the lead screw 160 and the nut 156.

The end of the distraction rod 106 opposite the end 110 including the fixation apertures or holes 120, 122, 124 is disposed within an inner surface of a tubular housing 126 of the adjustable portion 104. The distraction rod 106 is configured to telescope relative to the tubular housing 126. Despite the telescoping motion of the distraction rod 106 into and out of the tubular housing 126, the entry of foreign matter into the housing 126 may be prevented using one or more o-rings and other features as described in, e.g., U.S. Pat. No. 9,848,914.

The distraction rod 106 may be coupled to a magnetic assembly 136 via the lead screw 160 in combination with any of a number of torque transfer interfaces 300 as described herein.

The magnetic assembly 136, which is described in more detail below, acts as a driver to drive rotation. This rotation may be transmitted through a torque transfer interface 300 to a torque receiving component, or receiver as used herein. In the embodiments described herein, the receiver may typically be the lead screw 160.

The interface 300 between the lead screw 160 and the magnetic assembly 136 has several functions. The interface must withstand heavy compressive loads. It also may need to withstand large tensile loads. Furthermore, the interface must transmit torque from the rotating magnetic assembly 136 to the lead screw 160. The interface 300 must also maintain the concentric alignment between the lead screw 160 and the nut 156. With respect to compressive loads, these are transmitted down the lead screw 160 and into the magnetic assembly 136, which rides on a thrust ball bearing 150. With respect to tensile loads, these are transmitted from the magnetic assembly 136 across the interface 300 and up the lead screw 160. Torquing forces are transmitted from the magnetic assembly 136 to the lead screw 160 via the interface 300.

Because the magnetic assembly 136 may not deliver unlimited torque, even small mechanical losses due to component binding are desirable to avoid.

Aspects of the magnetic assembly 136 are best seen in FIG. 3. The magnetic assembly 136 includes an upper cup 140 and a lower cup 142. A permanent magnet 154 is located in the recess formed between the interior portions of the upper cup 140 and the lower cup 142. The permanent magnet 154 may be cylindrical in shape, and may be a radially poled magnet. The permanent magnetic 154 may have a diameter of, e.g., about 0.28 inches and a length of about 0.73 inches, although other dimensions may be used. The permanent magnet 154 may include, for example, a rare earth magnet formed from, for instance, Neodynium-Iron-Boron. The magnet may be made from a grade of N35 or higher, for example a grade of N50. The permanent magnet 154 is bonded or otherwise affixed to the upper cup 140 and the lower cup 142. This allows torque applied to the permanent magnet 154 to be transferred to the upper cup 140 and thus the lead screw 160. The permanent magnet 154 is shorter in length than the combined lengths of the internal cavities of the upper cup 140 and lower cup 142. This assures that when the magnetic assembly 136 is under compression, the upper cup 140 and the lower cup 142 are stressed, rather than stress being placed on the permanent magnet 154.

The permanent magnet 154 may be rotated or “driven” through the application of a magnetic field by an external adjustment device such as the external adjustment device described in U.S. Pat. No. 9,848,914. The external adjustment device may be placed against the subject's skin in the manner described therein, to remotely rotate the internal permanent magnet 154. As explained herein, rotation of the internal permanent magnet 154 causes rotational movement of the magnetic assembly 136. This rotational movement is then translated to the lead screw 160 via the interface 300. Depending on the rotational direction of the lead screw 160, the distraction rod 106 moves in a telescopic manner out of or into the adjustable portion 104 in response to the movement of the lead screw within the cavity 112. In this regard, by controlling the rotational movement of the magnetic assembly 136 using an external adjustment device, the operator is able to adjust the linear motion of the distraction rod 106 in a controllable manner.

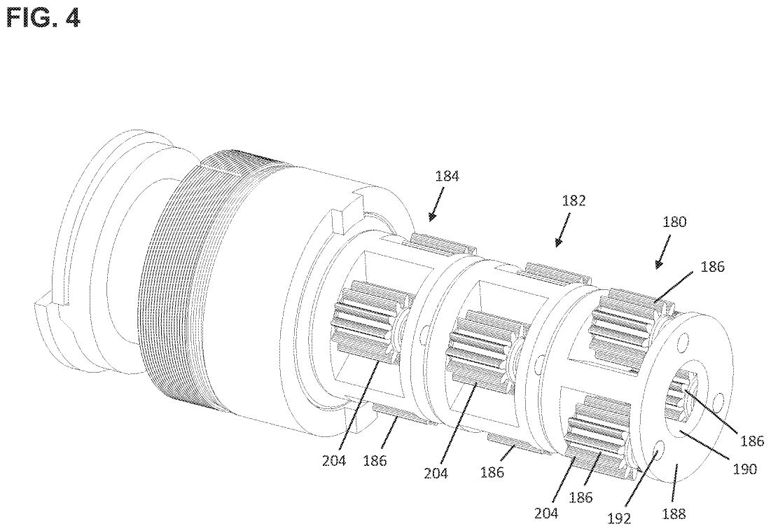

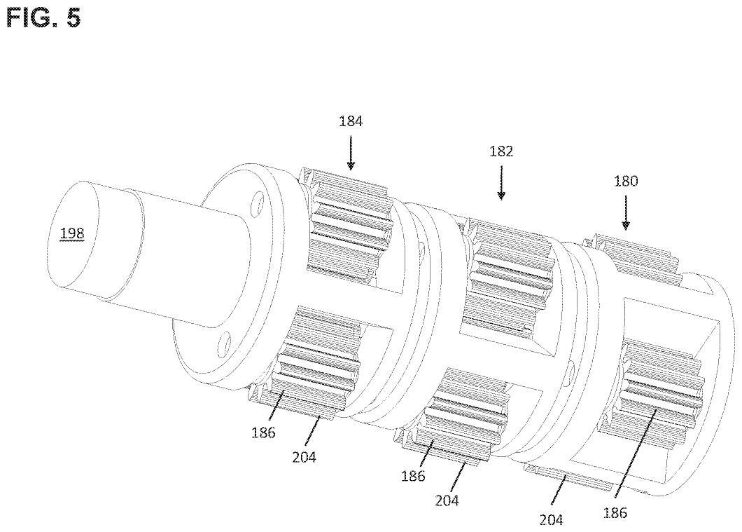

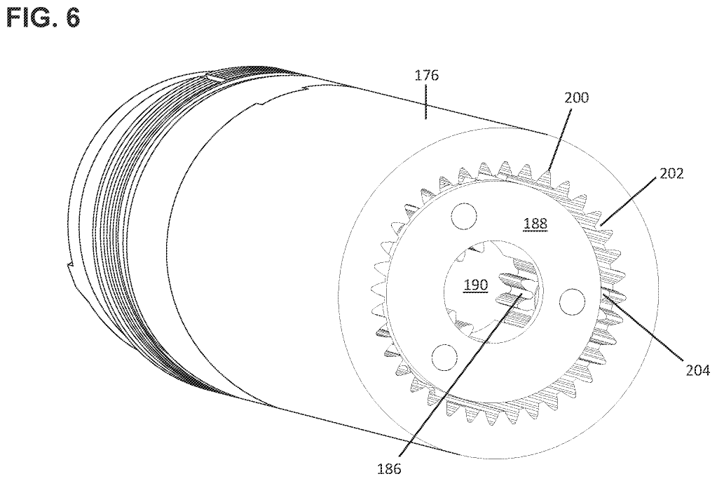

Returning to the magnetic assembly 136, the permanent magnet 154 may be contained within a magnet housing 164 having an end cap 166. The magnet housing 164 may further be welded to the end cap 166 to create a hermetic seal. The end cap 166 includes a cylindrical extension or axle 168 which fits within the inner diameter of a radial bearing 170, allowing for low friction rotation. The outer diameter of the radial bearing 170 fits within a cavity 172. The magnetic assembly 136 may terminate at an opposing end in a first sun gear 178 which may be integral to the magnet housing 164. Alternatively, the first sun gear 178 may be made as a separate component and secured to the magnet housing 164, for example by welding. In either case, the first sun gear 178 turns with rotation of the magnetic assembly 136 in a 1:1 fashion upon application of a moving magnetic field applied to the patient from an external location as discussed herein. As best shown in FIG. 4, the first sun gear 178 is configured to be inserted within an opening 190 in a first gear stage 180 having three planetary gears 186 which are rotatably held in a frame 188 by axles 192. A second sun gear 194 (see FIG. 3), which is the output of the first gear stage 180, turns with the frame 188. Identical components, including an opening 190, a frame 188, and three planetary gears 186 held by respective axles 192, are present in a second gear stage 182, which outputs to a third sun gear 196 (see FIG. 3), and the third gear stage 184, which also includes an opening 190, a frame 188, and three planetary gears 186 held by respective axles 192, like the first and second gear stages 180, 182. The third gear stage 184 outputs to an output shaft 198 as best seen in FIG. 5. As shown in FIG. 6, a ring gear 176 is disposed about the first through third gear stages 180, 182, 184, and extends axially along their length. The ring gear 176 includes internal teeth 202 along the inner wall 200 thereof. The internal teeth 202 of the ring gear 176 are configured to engage the externally extending teeth 204 of the planetary gears 186 as they turn. Each gear stage illustrated has a 4:1 gear ratio, so the output shaft 198 turns once for every 64 turns of the magnetic assembly 136. Because of the 64:1 gear ratio, the implant 100 is able to axially displace a bone segment against severe resisting forces, such as those created by soft tissue. A thrust bearing 262 (see FIG. 3) may be disposed between the lead screw 160 and the gear stages 180, 182, 184 in order to protect the gear stages 180, 182, 184 and the magnetic assembly 136 from compressive forces transmitted from the lead screw 160.

Torque is transferred from the driver to the receiver at the interface 300, as mentioned previously. Collectively, the magnetic assembly 136, the first, second, and third gear stages 180, 182, 184, and output shaft 198, and the components coupling these features to one another as described herein, may be considered the driver 302, while the lead screw 160 and features coupled thereto, and whose movement is fixed relative to the lead screw 160, may collectively be considered the receiver 304 (see FIG. 2). Thus, torque is generated by the magnetic assembly 136, amplified by the gear stages 180, 182, 184, output from the output shaft 198, and transferred by the interface 300 to the lead screw 160 and downstream driven or receiving components.

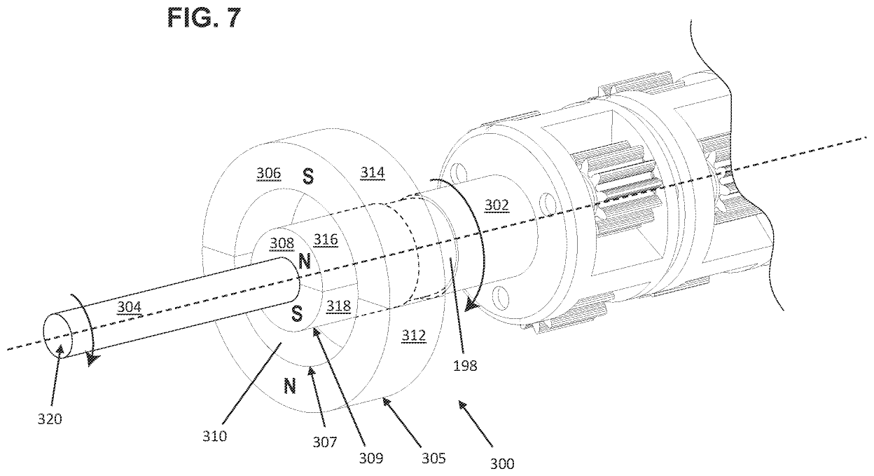

FIGS. 7-19 illustrate various interfaces 300 according to embodiments of the present disclosure. In certain embodiments, the interface 300 may be configured to transfer torque from the driver 302 to the receiver 304, while allowing axial translation of one or more components without compromising torque transfer. In certain embodiments, the interface 300 may further provide the advantages of facilitating side loading of components during assembly of the implant 100, rather than axial insertion. In still further embodiments, the interface 300 may be configured to provide a margin of float between the driver 302 and the receiver 304, allowing coupling and transfer of torque in spite of any imperfections that may be present in one component or another. Still further, in certain embodiments, the interface 300 may limit or avoid direct contact between the driver 302 and the receiver 304, and/or may adaptably receive non-concentric shafts. These features offer the advantages of reducing friction and wear, and improving efficiency of the torque transfer interface.

FIGS. 7-9 illustrate embodiments in which the interface 300 may be a magnetic interface, and may comprise a primary or first magnetic member 306 rotationally fixed to the driver 302, such that rotation of the driver 302 is configured to cause a rotation of the first magnetic member 306. The first magnetic member 306 may particularly be a dipole magnet, having a north pole 312 and a south pole 314. A secondary or second magnetic member is rotationally fixed to the receiver 304, such that a rotation of the second magnetic member 308 is configured to cause a rotation of the receiver 304. The first magnetic member 306 is configured to attract the second magnetic member 308, and the second magnetic member 308 is configured to rotate in response to a rotation of the first magnetic member 306.

Like the first magnetic member 306, in the embodiment of FIG. 7, the second magnetic member 308 may also include a dipole magnet, having a north pole 316 and south pole 318. The first magnetic member 306 and the second magnetic member 308 may be magnetically coupled to one another such that, e.g., the north pole 316 of the second magnetic member 308 attracts the south pole 314 of the first magnetic member 306, and the north pole 312 of the first magnetic member 306 attracts the south pole 318 of the second magnetic member 308.

With continued reference to FIG. 7, in certain embodiments, the second magnetic member 308 may be configured to rotate more slowly than the first magnetic member 306. For example, the rotation of the second magnetic member 308 may lag behind the rotation of the first magnetic member 306, in either speed or time of initiation of the rotational motion. In this manner, the relationship between first magnetic member 306 and second magnetic member 308 may soften the motion of the second magnetic member 308, such that even though the first magnetic member 306 rotates immediately upon rotation of the driver 302, the second magnetic member 308 may tend to seek ideal alignment over a brief period of time after the driver 302 has rotated. In this manner, the magnetic relationship between the first magnetic member 306 and the second magnetic member 308 may act as a shock absorber to limit the application of sudden changes in force to patient tissue, e.g., during a distraction procedure.

For bone movement processes such as, e.g., distraction, the optimum motion for bone growth is continuous, smooth, and slow. Torque transfer interfaces that employ a magnetic coupling between the driver 302 and the receiver 304 as described herein may therefore provide improved ability to achieve torque transfer with gradual motion, as the magnets transition from being out of phase to being in phase.

The first magnetic member 306 may have a cross sectional shape that is configured to provide a complementary fit with a cross sectional shape of the second magnetic member 308. In such embodiments, the second magnetic member 308 may be disposed at least partly within the first magnetic member 306 in a male to female arrangement, although the opposite arrangement may also be used. In either orientation, in certain embodiments, the first and second magnetic members 306, 308 may be substantially axially aligned with one another.

As illustrated in FIG. 7, in certain embodiments, the second magnetic member 308 may be disposed substantially or entirely within an opening or hollow space 310 within the first magnetic member 306. A certain amount, e.g., a fractional portion of a millimeter, of axial movement of the second magnetic member 308 relative to the first magnetic member 306 may be permitted. The first magnetic member 306 may be substantially annular, having a hollow cylinder or barrel shape, and may have an annular cross sectional geometry, while the second magnetic member 308 may have a solid cylindrical shape, and a circular cross sectional geometry. The first magnetic member 306 and the second magnetic member 308 may further be substantially concentrically arranged about an axis 320 of rotation. Due to the magnetic coupling between the first and second magnetic members 306, 308, the driver 302 and receiver 304 are rotationally locked with respect to one another. However, due to the opening of the hollow space 310 to the end of the first magnetic member 306 facing the receiver 304, the coupling between the first and second magnetic members 306, 308 permits axial movement at the interface 300 of the first magnetic member 306 relative to the second magnetic member 308. As illustrated in FIG. 7, in some embodiments, a space or gap may exist in the hollow space 310 between an inner wall 307 of the first magnetic member 306, and an outer wall 309 of the second magnetic member 308. In other embodiments, this space may be substantially eliminated, and the second magnetic member 308 may fit closely within the first magnetic member 306.

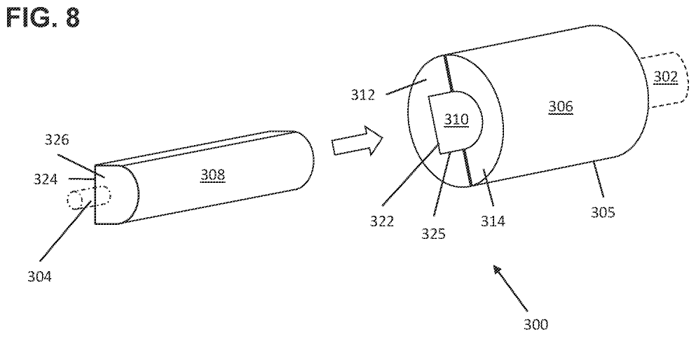

Turning next to FIG. 8, according to another embodiment, the first or primary magnetic member 306 is a dipole magnet having north and south poles 312 and 314, and a hollow space or opening 310 as discussed above relative to FIG. 7. Like the first magnetic member 306 of FIG. 7, the first magnetic member 306 of FIG. 8 may be substantially annular or may have a hollow cylindrical configuration, having a radially outward facing surface 305, and a hollow space or opening 310 therein. The hollow space 310 is open to at least the end of the first magnetic member 306 that faces the second magnetic member 308, and is configured to receive the second magnetic member 308 therein. The second magnetic member 308 may be disposed at least partly within the hollow space 310 within the first magnetic member 306, in a male to female arrangement. As illustrated in FIG. 8, the second magnetic member 308 may be inserted in the direction of the arrow into the hollow space 310 within the first magnetic member 306. In certain embodiments, the first and second magnetic members 306, 308 may be partially, substantially, or entirely axially aligned with one another, such that the second magnetic member 308 may be disposed partially, substantially, or entirely within the hollow space 310 disposed within the first magnetic member 306. Some axial movement the second magnetic member 308 may be permitted relative to the first magnetic member 306. In some embodiments, the hollow space 310 may be open to the end of the first magnetic member 306 that faces the driver 302 side, in addition to also being open to the end of the first magnetic member that faces the receiver 304 side). Such embodiments may permit a greater amount of axial movement of the second magnetic member 308 relative to the first magnetic member 306.

In the embodiment of FIG. 8, the second magnetic member 308 is made of a magnetic material. In certain embodiments, the second magnetic member 308 may not be a magnet itself, but may be, e.g., a ferrous metal bar. Although, as discussed above, the interface of FIG. 8 may permit axial movement of first and second magnetic member 306, 308 relative to one another, this axial movement may tend to be limited by the magnetic attraction between the first magnetic member 306 and the second magnetic member 308. This magnetic attraction may tend to contribute to the achievement and maintenance of axial alignment of the first and second magnetic members 306, 308 with respect to one another.

The first magnetic member 306, and particularly the hollow space 310 therein, may have a cross sectional shape 325 that is configured to provide a complementary fit with a cross sectional shape 326 of the second magnetic member 308. As shown in FIG. 8, the cross sectional shape 325 of the hollow space 310 in the first magnetic member 306 may include a first engagement surface 322, and the cross sectional shape 326 of the second magnetic member 308 may include a second engagement surface 324. The first engagement surface 322 is configured to engage the second engagement surface 324 when the second magnetic member 308 is disposed at least partly within the hollow space 310 of the first magnetic member 306. Thus, the second magnetic member 308 is configured to rotate in response to a force exerted by the first engagement surface 322 on the second engagement surface 324, upon a rotation of the first magnetic member 306. This engagement is configured to cause the second magnetic member 308 to rotate at a rate of speed equal to that of the first magnetic member 306.

As shown in FIG. 8, the second magnetic member 308 has a cross sectional shape 326 that is “D-shaped,” and the hollow space 310 has a corresponding and complementary cross sectional shape 325. Other geometries are also contemplated, such that the female cross sectional shape 325 and male cross sectional shape 326 may include a mated pair of, e.g., crescents, semi-circles, irregular shapes having at least one arc and at least one flat side, irregular shapes having at least one arc and at least two flat sides, irregular shapes having at least one arc and at least three flat sides, irregular shapes having at least one arc and at least four or more flat sides, a slotted circle or pair of separated semi-circles and corresponding flat face, a cross or Phillips head, hexalobular shapes, or any other mated pair of keyed shapes capable of transferring torque as described herein or known in the art.

In the embodiment of FIG. 8, although the mated pair of cross sectional shapes 326, 326 may be asymmetrical, each of the rotating features of the interface 300 is oriented in a concentric or substantially arrangement, having no material offset relative to the axis of rotation. The driver 302, the receiver 304, and the first and second magnetic members 306, 308 are in concentric or substantially relationship with one another. In some embodiments, the three-dimensional shape of the second magnetic member 308 may be conceptualized by beginning with an embodiment having the geometries depicted in FIG. 7, making a flat cut to remove an amount of material therefrom, and adding that material to an inner surface of the first magnetic member 306 in an inner surface thereof, so that the first and second magnetic members 306, 308 in aggregate form a cylinder in concentric arrangement with the driver 302 and receiver 304. In other embodiments, the flat surfaces forming, e.g., the engagement surface 324, may not be made of magnetic material. For example, the second magnetic member 308 may include a magnet surrounded by a covering or coating of a non-magnetic material such as steel that may contribute aspects of the cross sectional shape 326 and one or more engagement surfaces 324.

The first magnetic member 306 may be rotationally fixed to the driver 302, such that rotation of the driver 302 is configured to cause a rotation of the first magnetic member 306. The second magnetic member 308 is rotationally fixed to a receiver 304, such that a rotation of the second magnetic member 308 is configured to cause a rotation of the receiver 304. The second magnetic member 308 is configured to rotate in response to a rotation of the first magnetic member 306.

Due to the geometric relationship and magnetic attraction between the first and second magnetic members 306, 308 of FIG. 8, the driver 302 and receiver 304 are rotationally locked with respect to one another. However, in a manner similar to that described with respect to FIG. 7, the coupling between the first and second magnetic members 306, 308 permits axial movement at the interface 300 of the first magnetic member 306 relative to the second magnetic member 308.

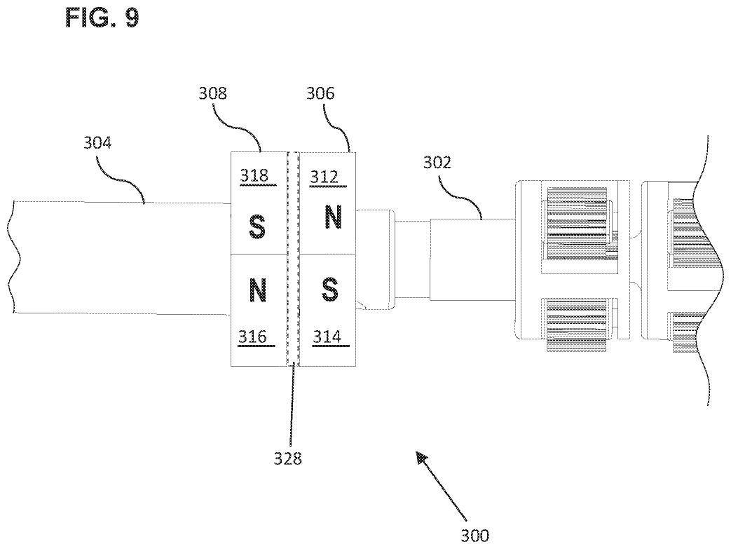

Turning next to the embodiment of FIG. 9, like that of FIG. 7, the interface 300 may include a first magnetic member 306 that includes a dipole magnet having a north pole 312 and a south pole 314, and a second magnetic member 308, which may include a dipole magnet having a north pole 316 and south pole 318. The first magnetic member 306 and the second magnetic member 308 may be magnetically coupled to one another such that, e.g., the north pole 316 of the second magnetic member 308 attracts the south pole 314 of the first magnetic member 306, and the north pole 312 of the first magnetic member 306 attracts the south pole 318 of the second magnetic member 308. As shown in FIG. 9, each of the first magnetic member 306 and the second magnetic member 308 may comprise a disc-shaped dipole magnet, which may be coupled to the driver 302 and the receiver 304, respectively.

The embodiment of FIG. 9 may further include a friction-reducing component or thrust bearing 328 disposed at an axial position between the first magnetic member 306 and the second magnetic member 308, configured to prevent the first and second magnetic members 306, 308 from contacting one another, and to resist damage to the magnetic members 306, 308 in the event that the magnetic members 306, 308 are forced together.

With continued reference to the embodiment of FIG. 9, in certain embodiments, the second magnetic member 308 may be configured to rotate more slowly than the first magnetic member 306, in a manner similar to that described above relative to the embodiment of FIG. 7. In some examples, the final gear stage is linked to the first round magnet (right most) and the lead screw is linked to the second round magnet (left most). For example, the rotation of the second magnetic member 308 may lag behind the rotation of the first magnetic member 306, in either speed or time of initiation of the rotational motion. In this manner, the relationship between first magnetic member 306 and second magnetic member 308 may soften the motion of the second magnetic member 308, such that even though the first magnetic member 306 rotates immediately upon rotation of the driver 302, the second magnetic member 308 may tend to seek ideal alignment over a brief period of time after the driver 302 has rotated. In this manner, the magnetic relationship between the first magnetic member 306 and the second magnetic member 308 may act as a shock absorber to limit the application of sudden changes in force to patient tissue, e.g., during a distraction procedure.

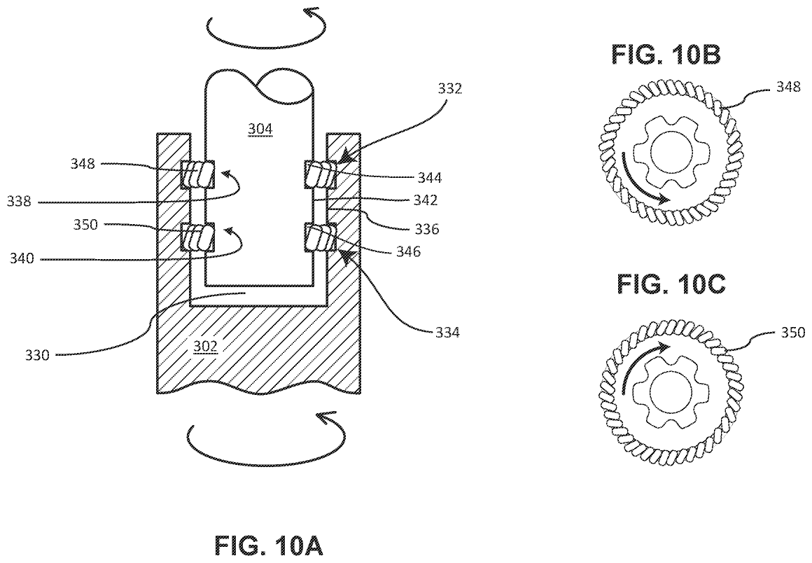

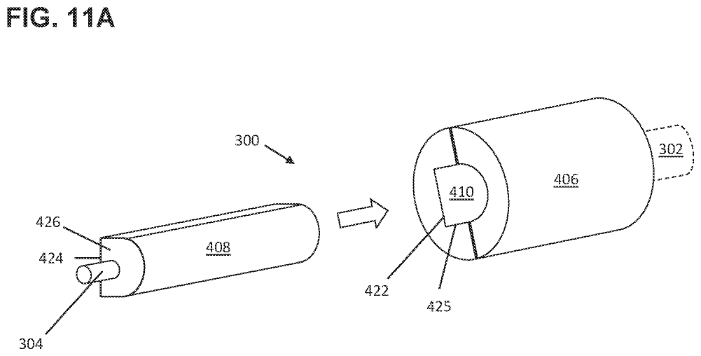

FIGS. 10A-10C illustrate another embodiment of an interface 300 that is configured to transfer torque from a driver 302 to a receiver 304. As shown in the embodiment of FIG. 10A, the interface 300 includes a receptacle 330 disposed on an end of the driver 302, that is open to the end of the driver 302 that interfaces with the receiver 304. The receptacle 330 is configured to accommodate an end portion of the receiver 304 therein. A first receptacle groove 332 and a second receptacle groove 334 are each disposed on an annular inner side wall 336 of the receptacle 330. A first receiver groove 338 and a second receiver groove 340 may each be disposed on a radially outer surface 342 of the end portion of the receiver 304. The pair of grooves made up of the first receptacle groove 332 and the first receiver groove 338 are axially aligned such that when the end portion of the receiver 304 is disposed within the receptacle 330, the first receptacle groove 332 and the first receiver groove 338 mate to form a first annular space 344. In a similar manner, the pair of grooves made up of the second receptacle groove 334 and the second receiver groove 340 are axially aligned such that when the end portion of the receiver 304 is disposed within the receptacle 330, the second receptacle groove 334 and the second receiver groove 340 mate to form a second annular space 346. The first and second annular spaces 344 and 346 are axially spaced from one another along the axial extent of the interface between the receptacle 330 and the end portion of the receiver 304.

A first toroidal spring 348 is disposed in the first annular space 344, such that it is disposed circumferentially about the end portion of the receiver 304. The first toroidal spring 348 is canted in a first direction as shown in FIG. 10B. A second toroidal spring 350 is disposed in the second annular space 346, also disposed circumferentially about the end portion of the receiver 304. The second toroidal spring is canted in a second direction that is opposite the first direction, as shown in FIG. 10C. In certain embodiments, the directions may be opposite, such that the first toroidal spring 348 is canted in a second direction as shown in FIG. 10C, and the second toroidal spring 350 is canted in a first direction that is opposite the second direction, as shown in FIG. 10B.

When used together as shown in FIG. 10A, first and second coil springs 348, 350 act as an infinite ratchet. Rotation of the driver 302 in one direction causes the coils of one spring 348 or 350 to lay down, while rotation of the driver 302 in the opposite direction causes the coils of the other of the springs 348 or 350 to stand up, resisting movement. As described above with respect to FIGS. 10B and 10C, coil springs 348 and 350 are inserted in opposite directions, such that they are configured to self-jam and locking rotational movement of the receiver 304 which is disposed through the coil springs 348, 350. This interaction permits the transfer of torque from the driver 302 to the receiver 304, while offering advantageous ease of assembly. In particular, the receiver 304 is simply axially inserted through the coil springs 348, 350, limiting the need to precisely align the driver 302 and receiver 304. Additionally, this arrangement provides some flexibility, i.e. float or play, to the coupling between the driver 302 and the receiver 304, bridging any potential imperfections in the driver 302, the receiver 304, and/or the alignment between the driver 302 and receiver 304 within the implant 100.

In some embodiments, the first toroidal spring 348 and the second toroidal spring 350 may comprise a coil spring having a first end thereof welded or otherwise affixed to a second end thereof. The coil springs 348, 350 may be made of, e.g., stainless steel, or more particularly of 17-7 stainless steel.

In certain embodiments, such as the one shown in FIG. 10A, the first toroidal spring 348 and the second toroidal spring 350 may have the same diameter as the other coil spring. In other embodiments, the first toroidal spring 348 has a first diameter, and the second toroidal spring 350 has a second diameter that is different than the first diameter. In such an embodiment, the first end of the receiver 304 may include a stepped diameter shaft, with the first annular space 344 disposed at a first step and having the first diameter, and the second annular space 346 disposed at a second step and having the second diameter. In a further embodiment, the end portion of the receiver 304 may further comprise a shaft and a collar disposed thereon. The combination of the shaft and collar may achieve the effect of a stepped diameter shaft. In such an embodiment, one of the first annular space 344 or the second annular space 346 may be disposed on the collar, and the other of the first annular space 344 or the second annular space 346 may be disposed on the shaft.

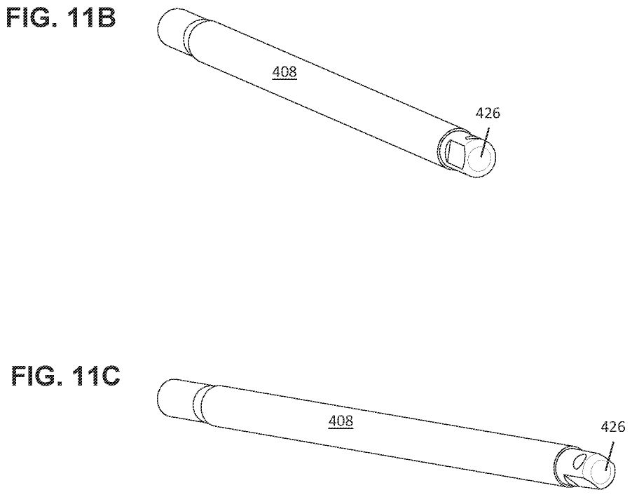

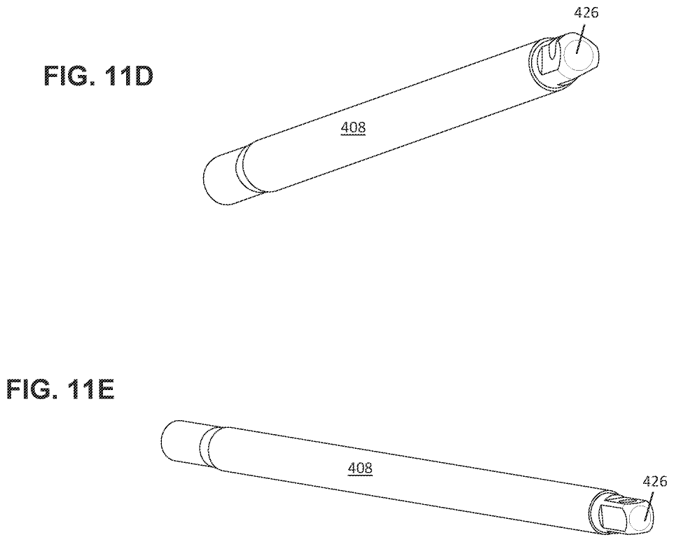

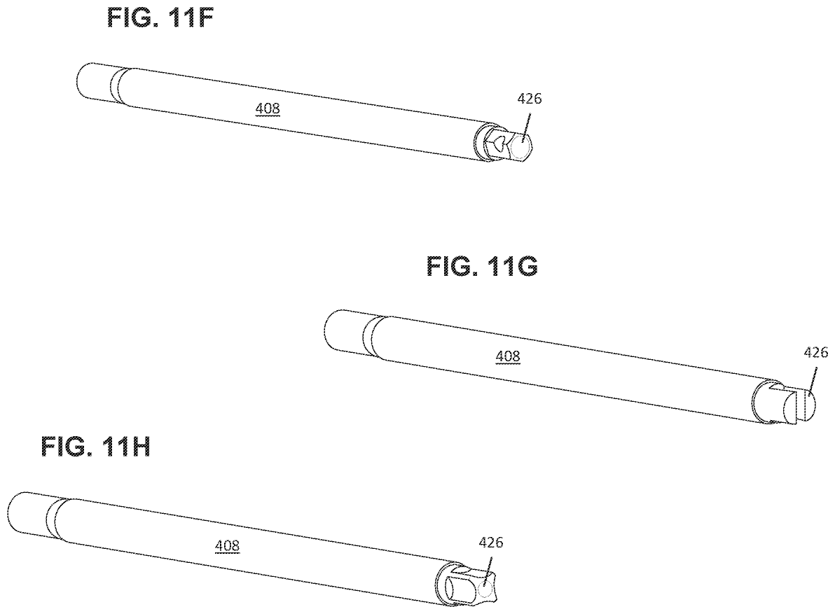

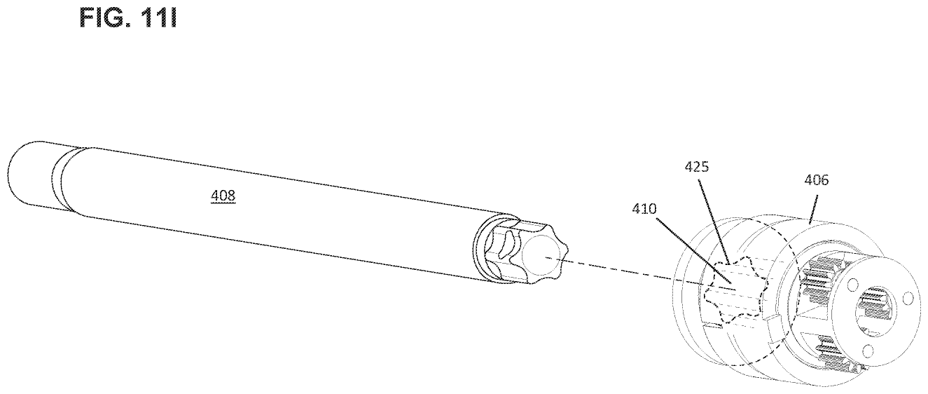

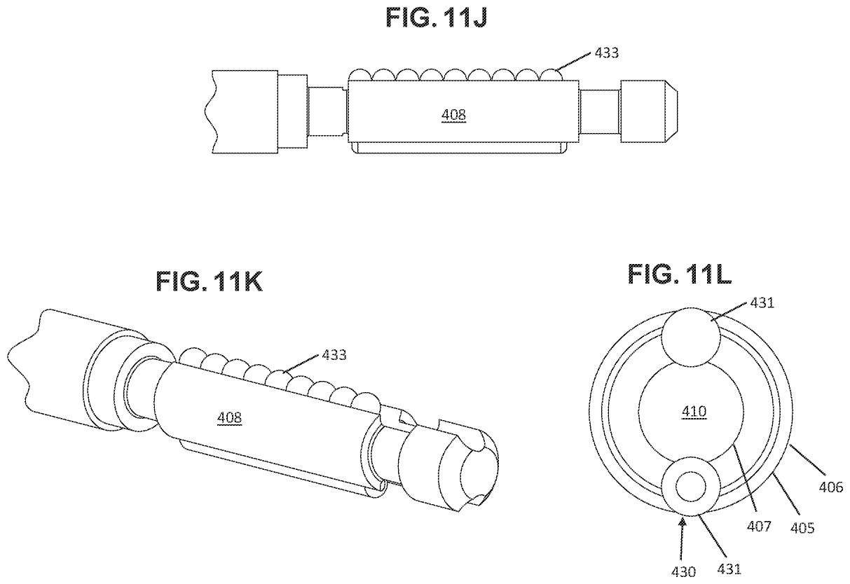

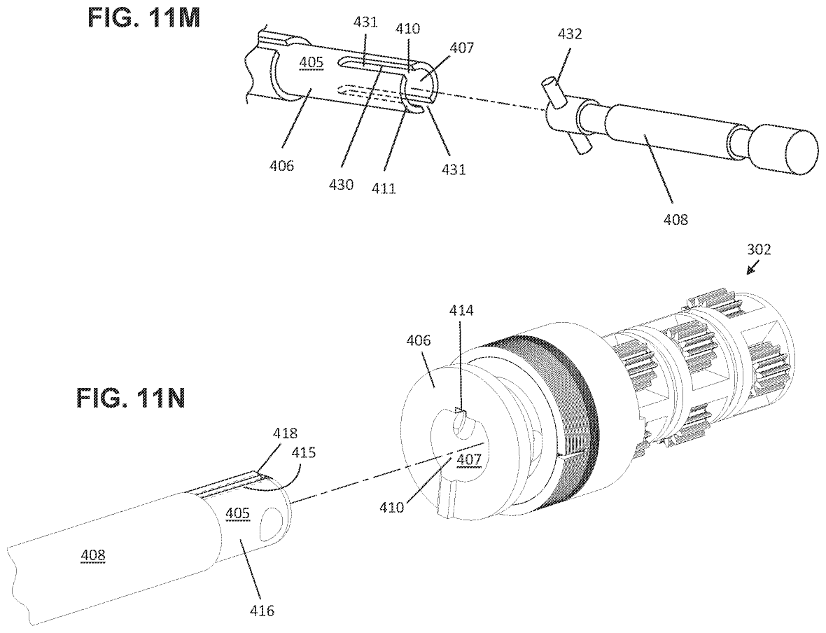

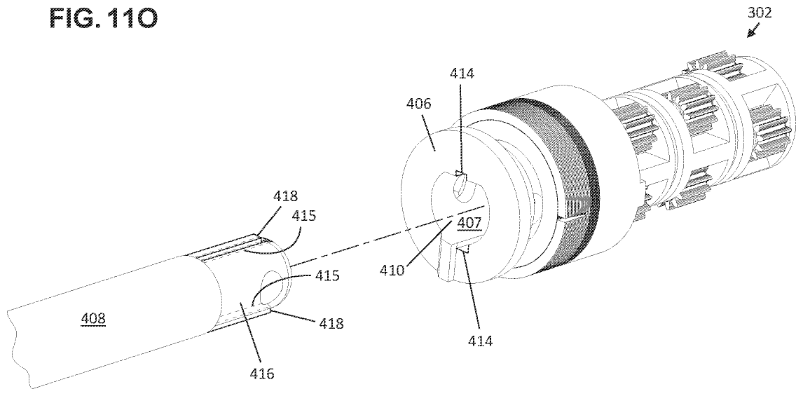

Turning next to FIGS. 11A-11O, a variety of interfaces 300 are provided that include any of a number of complementary geometric features configured to translate rotation from a driver 302 to a receiver 304. Such interfaces 300 may be configured to include geometric features similar to those of FIG. 8 as described above. However, in interface 300 as illustrated in FIGS. 11A-11O, the first member 406, and male member 408 may be made of non-magnetic materials.

In the embodiment of FIG. 11A, the female member 406 may include a hollow space 410 therein, which may have a cross sectional shape 425 that is configured to provide a complementary fit with a cross sectional shape 426 of the male member 408. The cross sectional shape 425 of the hollow space 410 in female member 406 may include a first engagement surface 422, and the cross sectional shape 426 of the male member 408 may include a second engagement surface 424. The first engagement surface 422 is configured to engage the second engagement surface 424 when the male member 408 is disposed at least partly within the hollow space 410 of the female member 406, upon rotation of the female member 406 in response to rotation of the driver 302.

In various embodiments described herein, more than one engagement surface 422, 424 may be present on the cross sectional shapes 425, 426 of the male member 408 and the hollow space 410 of the female member 406. Regardless of the number of engagement surfaces, the male member 408 is configured to rotate in response to a force exerted by the first engagement surface 422 on the second engagement surface 424. This engagement is configured to cause the male member 408 to rotate at a rate of speed equal to that of the female member 406.

As shown in FIG. 11A, the male member 408 may have a cross sectional shape 426 that is “D-shaped,” including an arc and a flat surface (see also FIG. 11B), and the hollow space 410 has a corresponding and complementary cross sectional shape 425. Other geometries are also contemplated, as illustrated in FIGS. 11C through 11O. Each of FIGS. 11B through 11I, 11N, and 11O illustrates an exemplary shape which may be used as the cross sectional shape 426, which may be configured to matingly engage with a corresponding and complementary cross sectional shape 425 of hollow space 410 in the female member 406 (FIGS. 11A, 11I). The female cross sectional shape 425 and male cross sectional shape 426 may include any now known or later developed mated or keyed shapes capable of transferring torque, including, for example: a crescent, semi-circle, or “D-shape” (FIG. 11B), an irregular shape having two flat surfaces and one or more arced surfaces (FIG. 11C), an irregular shape having three flat surfaces and one or more arced surfaces (FIG. 11D), an irregular shape having four flat surfaces and one or more arced surfaces (FIG. 11E), a regular or irregular hexagon (FIG. 11F), a slotted circle (FIG. 11G) mated with a flat blade feature, a cross or Phillips head (FIG. 11H), a hexalobe (FIG. 11I), or keyed geometries (FIGS. 11J, 11K, 11L, 11M, 11N, and 11O). Any of the foregoing geometric features may be formed as an extrusion of the indicated geometric shape to form the male member 408. The male member 408 may then be disposed within a complementary shaped hollow space or opening 410 within the female member 406. As discussed relative to certain other embodiments, e.g., those of FIGS. 7 and 8, the geometric engagement of male member 408 and female member 406 rotationally locks the driver 302 to the receiver 304, but permits axial movement of the male member 408 and female member 406 relative to one another.

As shown in FIGS. 11J-11M, a guide ball(s) (FIGS. 11J, 11K, 11L) or guide pin(s) (FIG. 11M) may be used to transfer torque from a driver to a receiver. In such embodiments, the female member 406 (FIG. 11L) includes an axial slot guide 430. The axial slot guide 430 includes at least one slot 431 (FIG. 11M) extending radially through the thickness of the first member 406, from the inner wall 407 defining the hollow space 410 to the radially outward facing surface 405. In certain embodiments, the axial slot guide 430 includes two such slots 431, each spaced about 180 degrees apart from the other, with respect to the diameter of the female member 406.

The axial slot guide 430 including slot(s) 431 extends axially from the end surface 411 (FIG. 11M) of the female member 406 that interfaces with the male member 408, to a position partway along the axial extent of the female member 406. The male member 408 may include a ball feature 433 (FIGS. 11J-11L) or a pin feature 432 (FIG. 11M) which extends radially outward from the male member 408 in a direction perpendicular to the axis of rotation. Ball(s) 433 or pin 432 may be positioned near the end of the male member 408 which interfaces with the female member 406 and/or along an axial length of the male member 408. In embodiments including only one slot 431 in the female member 406, the ball(s) 433 or pin 432 may extend radially outward from the male member 408 in one direction. In embodiments including two slots 431 as shown in FIG. 11M, a pair of pins 432 may extend radially outward in opposite directions from the male member 408. Other arrangements are also possible, such as those in which the pins 432 and slots 431 engage at different angular relationships such as, e.g., 90 degrees, in which balls 433 extend in more than one radial direction, etc.

In use, the male member 408 is inserted into the hollow space or opening 410 in female member 406, and rotationally positioned such that the ball(s) 433 or pin(s) 432 are aligned with the slot(s) 431 in the axial slot guide 430. With the ball(s) 433 or pin(s) 432 aligned with slot(s) 431, the male member 408 may be inserted into female member 406. The ball(s) or pin(s) 432 fit closely within the width of the slot(s) 431, such that when the driver 302 rotates the female member 406, the wall of the slot(s) 430 exerts a rotational force on the ball(s) 433 or pin(s) 432 disposed therein, and causes the male member 408 to rotate, carried by the ball(s) 433 or pin(s) 432. The closer the fit between the ball(s) 433 or pin(s) 432 and the walls of the slot(s) 431, i.e., the smaller the gap between the ball(s) 433 or pin(s) 432 and the walls of the slot(s) 431, the more immediate and precise the torque transfer will be.

Turning next to FIG. 11N, an embodiment of an interface 300 is illustrated in which a keyway 414 and a key 418 are used to rotationally lock the driver to the receiver to transfer torque. In such an embodiment, the female member 406 may be in the form of a sleeve affixed to the driver, and may include a hollow space 410 therein, open to the end opposite the connection to the driver 302.

The hollow space 410 may have a substantially circular cross sectional shape, configured to receive the male member 408. At one or more positions along the circumference of the inner wall 407 of the hollow space or opening 410, the opening may further include a keyway 414 axially extending along the opening 410, and fluidly connected with the portion of the opening 416 having a substantially circular cross-sectional shape. The keyway 414 may be in the form of, e.g., a groove, channel, or other recessed shape disposed on the inner wall 407.

The male member 408 may have a substantially circular cross sectional shape, and may be configured to be received within the opening 410 of the female member 406. At one or more positions along the circumference of the radially outer surface 405 of the male member 408, the male member 408 may include a keyway 415 axially extending along the outer surface 405. The keyway 415 may be in the form of, e.g., a groove, channel, or other recessed shape, and may correspond in shape and dimension to the keyway 414 on the opening 410. When the male member 408 is inserted into the opening 410, the male member 408 may freely rotate within the opening 410 in the absence of a key.

In use, keyways 414, 415 may be rotationally aligned with one another. In this position, as shown in FIG. 11N, keyways 414 and 415 together form a single keyway in which a key 418 may be disposed. A key 418 may be placed with one end in the keyway 414 of the female member 406, and the other end in the keyway 415 of the male member 408. Once the key 418 is situated within both keyways 414, 415, the male member 408 and female member 406 may translate relative to one another, but are rotationally locked with respect to each other by the presence of key 418 in the keyways 414, 415. The closer the fit between the key 418 and the keyways 414, 415, i.e., the smaller the gap between the key 418 and the keyways 414, 415, the more immediate and precise the torque transfer will be.

FIG. 11O depicts an interface 300 similar to that of FIG. 11N. In embodiment of FIG. 11O, the interface 300 includes a pair of keyways 414 and a pair of keys 418 to rotationally lock the driver to the receiver to transfer torque. In such an embodiment, the female member 406 may be in the form of a sleeve affixed to the driver, and may include a hollow space 410 therein, open to the end opposite the connection to the driver 302.

The hollow space 410 may have a substantially circular cross sectional shape, configured to receive the male member 408. At one or more positions along the circumference of the inner wall 407 of the hollow space or opening 410, the opening may further include a first keyway 414 axially extending along the opening 410, and fluidly connected with the portion of the opening 416 having a substantially circular cross-sectional shape. The keyway 414 may be in the form of, e.g., a groove, channel, or other recessed shape disposed on the inner wall 407. A second keyway 414 may axially extend along the opening 410, fluidly connected with the portion of the opening 416 having a substantially circular cross-sectional shape. The two keyways 414 may be radially spaced relative to one another, e.g., about 90 degrees, about 180 degrees (as shown in FIG. 11O), about 270 degrees, or other measure intermediate between the foregoing exemplary angular measurements. Like the first keyway 414, the second keyway 414 may also be in the form of, e.g., a groove, channel, or other recessed shape disposed on the inner wall 407.

The male member 408 may have a substantially circular cross sectional shape, and may be configured to be received within the opening 410 of the female member 406. At one or more positions along the circumference of the radially outer surface 405 of the male member 408, the male member 408 may include a pair of keyways 415 axially extending along the outer surface 405. The keyways 415 may be in the form of, e.g., a groove, channel, or other recessed shape, and may correspond in shape and dimension to the keyway 414 on the opening 410. The two keyways 415 may be radially spaced relative to one another, e.g., about 90 degrees, about 180 degrees (as shown in FIG. 11O), about 270 degrees, or other measure intermediate between the foregoing exemplary angular measurements. When the male member 408 is inserted into the opening 410, the male member 408 may freely rotate within the opening 410 in the absence of keys 418.

In use, the pairs of keyways 414, 415 may be rotationally aligned with one another. In this position, as shown in FIG. 11O, keyways 414 and 415 together form a single keyway in which a key 418 may be disposed. A key 418 may be placed with one side in the keyway 414 of the female member 406, and the other side in the keyway 415 of the male member 408. Once the key 418 is situated within both keyways 414, 415, the male member 408 and female member 406 may translate relative to one another, but are rotationally locked with respect to each other by the presence of the key 418 in the keyways 414, 415. The closer the fit between the key 418 and the keyways 414, 415, i.e., the smaller the gap between the key 418 and the keyways 414, 415, the more immediate and precise the torque transfer will be.

Regardless of which embodiment of, e.g., FIGS. 11A-11O is employed, due to the geometric relationships between the female and male members 406, 408, the driver 302 and receiver 304 are rotationally locked with respect to one another. In the embodiments of FIGS. 11A-11O, regardless of the symmetry or asymmetry of the cross sectional shape of a particular male member 408 and corresponding opening 410, each of the rotating features of the interface 300 is oriented in a substantially concentric arrangement with no offset relative to the axis of rotation. The driver 302, the receiver 304, and the female and male members 406, 408 are in concentric relationship with one another.

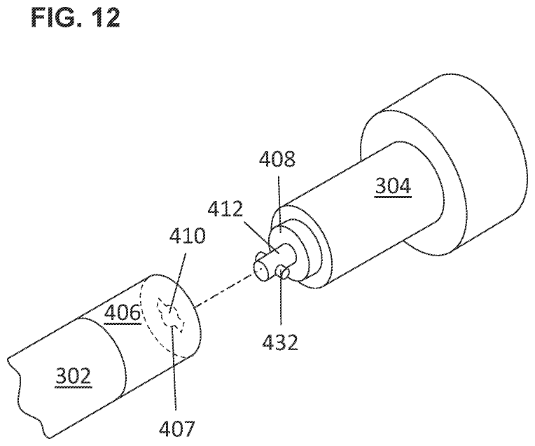

FIG. 12 illustrates an embodiment of an interface 300 utilizing a pin and shank sleeve coupling. In such an embodiment, the female member 406 may be in the form of a sleeve affixed to the driver 302, and may include a hollow space 410 therein, open to the end opposite the connection to the driver 302. The hollow space 410 may have a cross sectional shape that is configured to provide a complementary fit with a cross sectional shape of a portion of the male member 408. At one end, opposite the end at which the male member is coupled to the receiver 304, the male member 408 may include a shank 412 having a pin 432 extending in a direction perpendicular to the axis of rotation. Female member 406 may include an opening 410 having a cross sectional shape configured to matingly receive the shank 412 and pin 432 therein. The pins 432 may be disposed at a point along the axial extent of the shank 412, such that in use, the end of the shank 412 may be inserted into the opening 410 regardless of rotational orientation, but insertion of the pins and portion of the shank beyond the pins 432 requires rotational alignment of the pins 432 with the profile of the opening 410. With the pin(s) 432 and the opening 410 aligned, the shaft 412 and pins 432 of male member 408 may be inserted into the opening 410 of the female member 406. The pin(s) 432 fit closely within the cross sectional shape of the opening 410, such that when the driver 302 rotates the female member 406, the inner wall 407 of the opening 410 exerts a rotational force on the pin(s) 432 disposed therein, and causes the male member 408 to rotate, carried by the pin(s) 432. The closer the fit between the pin(s) 432 and the walls 407 of the opening 410, i.e., the smaller the gap between the pin(s) 432 and the walls 407 of the opening 410, the more immediate and precise the torque transfer will be.

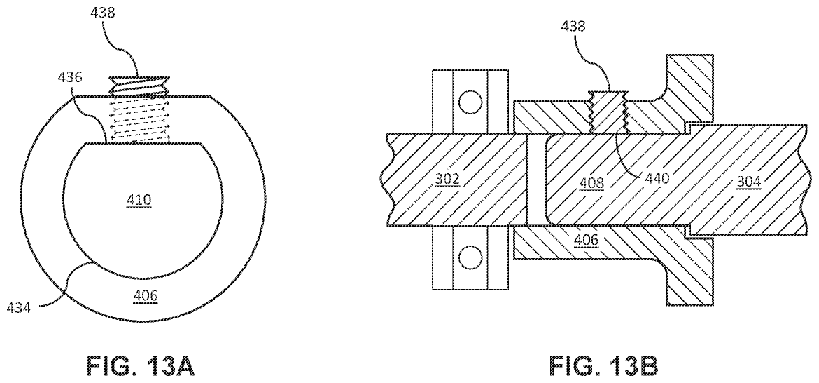

FIGS. 13A and 13B illustrate an embodiment of an interface 300 utilizing a set screw and a rod having a non-circular cross-sectional shape, for example, a D-shape, to rotationally lock the driver 302 to the receiver 304 to transfer torque. In such an embodiment, the male member 408 may have a non-circular cross-sectional shape having at least one arced surface 434 and at least one flat surface 436. In the embodiment illustrated in FIG. 13A, the male member 408 may include a D-shaped cross-sectional shape, similar to FIG. 11B. The female member 406 may be in the form of a sleeve affixed to the driver, and may include a hollow space 410 therein, open to the end opposite the connection to the driver 302. Unlike the embodiment of FIG. 11A, in which the female member 406 has a round outer diameter, and only the cross sectional shape of the opening 410 corresponds to the cross sectional shape of the male member 408, in FIG. 13A, the female member 406, as well as the opening 410 therein have a cross sectional shape that includes at least one arced surface and at least one flat surface, corresponding to the cross sectional shape of the male member 408, including the arced surface 434 and the flat surface 436. The opening 410 is configured to receive male member 408 therein. Due to the engagement between the inner surface of the opening 410 and the outer surface of the male member 408, the male and female members 406, 408, and the driver 302 and receiver 304 respectively coupled thereto, are rotationally locked to one another, and capable of transferring torque from driver to receiver, in a manner similar to that described relative to FIGS. 8 and 11A-B. Once the male member 408 is inserted to the desired axial position within the opening 410, a set screw 438 may be inserted within an aperture 440 in the flat surface of the female member 406, thereby locking the axial position of the male member 408 relative to the female member 406.

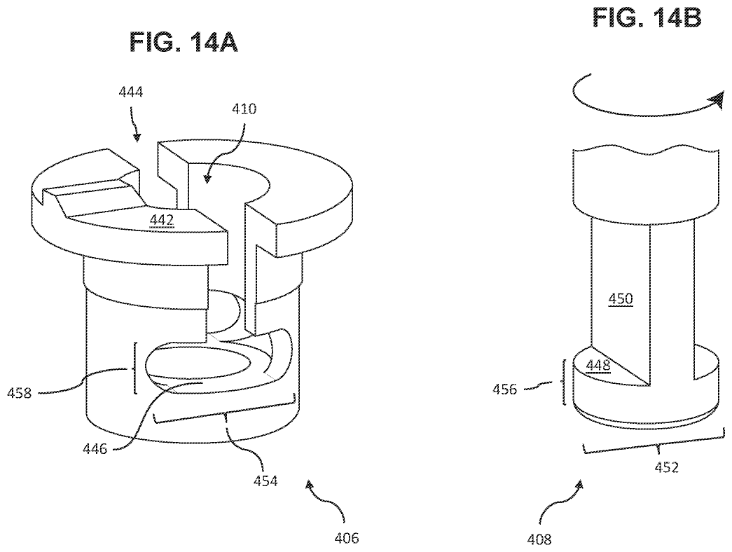

FIGS. 14A-14B illustrate an embodiment of an interface 300 utilizing a T-slot to rotationally lock the driver to the receiver to transfer torque. In such an embodiment, the torque receiver may include a male member 408 disposed on an end thereof, configured to interface with a corresponding and complementary female member 406 coupled to the driver. In some embodiments, the male member 408 may be disposed on an end of the lead screw 160. As shown in FIG. 14B, the male member 408 may include a feature having a T-shaped cross section, including a disk-shaped member 448 coupled to the receiver by an axially extending shaft 450.

As shown in FIG. 14A, the female member 406 may be in the form of a substantially cylindrical member 442 affixed at one end to the driver and may include an opening 410 therein that is open to the end opposite the connection to the driver 302. The opening 410 may include a T-slot configured to complement and receive the T-shaped feature of male member 408. To make up the T-slot, the opening 410 may include an axially extending slot portion 444 that is open at one end to the end of the female member 406 that interfaces with a complementary male member 408, and at the other end, to a lateral slot portion 446. The lateral slot portion 446 is perpendicular to the axially extending slot portion 444, and extends across the axis of rotation of the female member 406. The lateral slot portion 446 is open at one or both ends of the slot portion 446, i.e., across the diameter of the female member 406. This allows the male member 408 having the T-shape configuration to be laterally inserted into the female member 406. This configuration is in contrast with certain other configurations described herein, in which the male member 308, 408 is axially inserted into the female member 306, 406 (see, e.g., FIGS. 7, 8, 10A-10C, 11A-11O, and others). Once the male member 408, including the disc 448 and the shaft 450, is laterally inserted into the T-shaped slot including the axially and laterally extending portions 444, 446, further lateral movement of the male member 408 is constrained by circumferentially arranged components in the implant. The male member 408 is also limited in its axial movement by the axial constraints placed on movement of the disc 448 by the walls of lateral slot portion 446. The closer the fit between the width 452 of the disc 448 and the width 454 of the lateral slot portion 446, the smaller the gap between the disc 448 and the walls of the lateral slot portion 446, and the less lateral movement will be permitted. Less lateral movement will result in more immediate and precise the torque transfer will be from the driver to the receiver. The closer the fit between the thickness 456 of the disc 448 and the depth 458 of the lateral slot portion 446, the smaller the axial gap between the disc 448 and the walls of the lateral slot portion 446, and the less axial movement will be permitted.

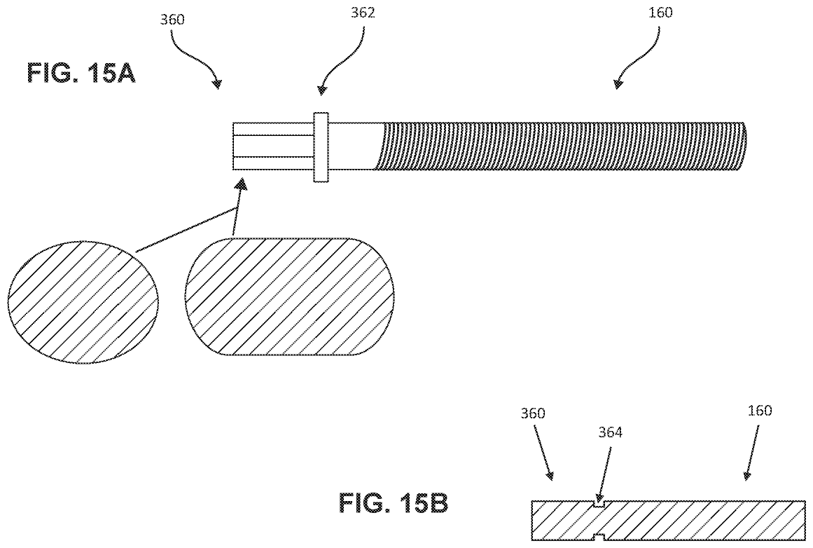

FIGS. 15A-15B illustrate an embodiment of an interface 300 utilizing a spline 360 (FIGS. 15A-15B) and retaining ring 362 (FIG. 15A) to rotationally lock the driver to the receiver to transfer torque. In such an embodiment, the interface 300 includes the spline 360 at one end and a lead screw 160 at the opposite end. The interface 300 can transfer torque therebetween.

The spline 360 can be a specially formed section of the interface 300 component or a fixture coupled thereto. The spline can effectively change the cross-sectional shape of the torque receiving or transferring end of the lead screw 160 to have a non-circular cross section so as to facilitate the transfer of torque to or from another component. In the illustrated embodiment, the spline 360 can have an oval shape in cross section configured to mate with an oval shaped receiver. Also illustrated is a spline 360 having a rounded shape with two flats that mate with another part having complementary features. The shape of the spline 360 in cross section can contribute to torque transfer. As shown in FIG. 15B, a recess 364 may be disposed between the spline 360 and lead screw 160, which may be configured to receive a retaining ring 362 (FIG. 15A). The retaining ring 362 may be configured to retain the relative positions of the moving parts.

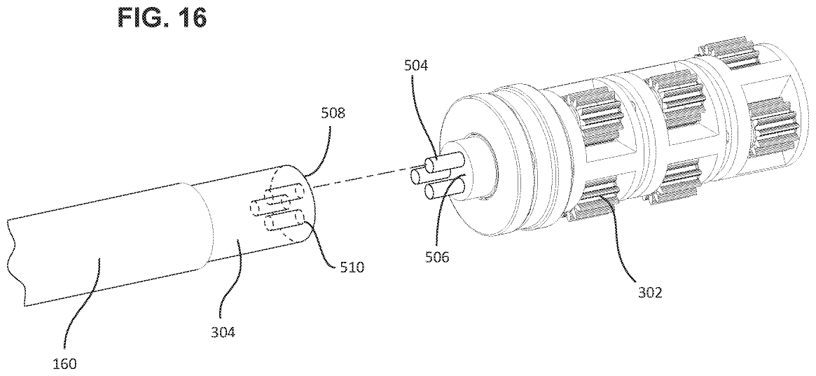

FIG. 16 illustrates an embodiment of an interface 300 utilizing a three-pin coupling to rotationally lock the driver to the receiver to transfer torque. In such an embodiment, a first member 506 is coupled at one end to the driver 302, for example, the output of a final gear stage. At the other end, the first member 506 is configured to interface with the receiver, e.g., a lead screw 160. The first member 506 may include three pins 504 extending axially from the end configured to interface with the end of the lead screw 160. The three pins 504 may be arranged about the end of the first member 506 such that they are equidistant from one another, and are symmetrical in their arrangement. The lead screw 160 may include a set of three holes 510 disposed on an end surface 508 of the lead screw 160, each hole 510 being configured to receive a corresponding pin 504. More or fewer pins 504 and corresponding holes 510 may be used, however symmetry may be maintained regardless of the number of pins 504 and holes arranged about the end surface of the first member 506 and the end surface 508 of the lead screw 160. The pins 504 may be axially inserted into the holes 510, thereby rotationally locking the first member 506 to the lead screw 160, thereby transferring torque from the driver to the lead screw. The closer the fit of the pins 504 within the holes 510, the more immediate and precise the transfer of torque will be.

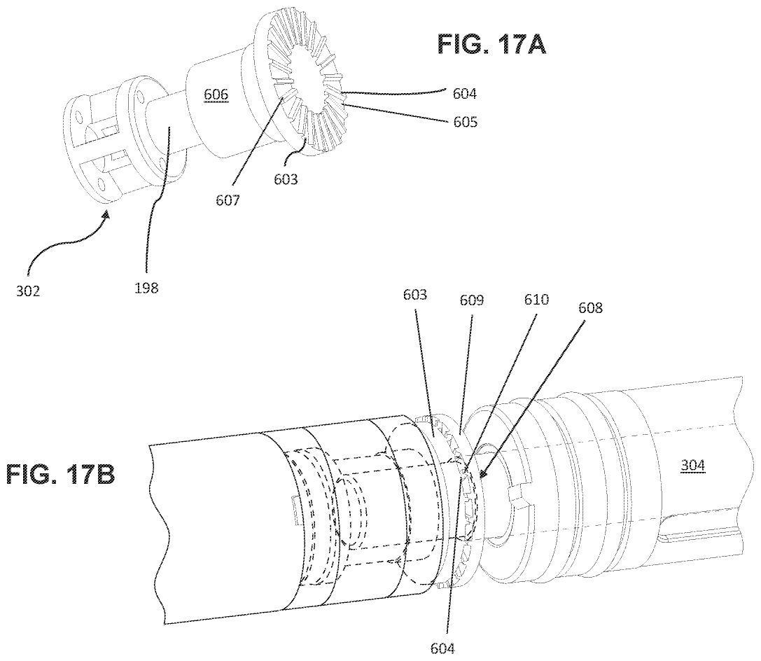

FIG. 17 illustrates an embodiment of an interface 300 utilizing plates having radially disposed ridges on mating faces to rotationally lock the driver to the receiver to transfer torque. In such an embodiment, a first member 606 is coupled at one end to the driver 302, for example, to the output 198 of a final gear stage. At the other end, the first member 606 includes an annular plate 603 having a plurality of ridges 604 disposed on the end face of the annular plate 603. A second member 608 may be coupled on one end to the torque receiving component 304, and may on the opposite end, include an annular plate 609 having a plurality of ridges 610 disposed on the end face of the annular plate 609. The ridges 604 and 610 are configured to engage one another to transmit torque.

The ridges 604 disposed on the plate 603 extend radially outward on the end face of the annular plate 603, with valleys 605 between each radially extending ridge 604. The ridges 604 may be spaced from each other at regular intervals around the circumference of the annular plate 603. In some embodiments, the ridges 604 may include chamfers 607 on one edge of each of the ridges 604, in the same direction. The ridges 604 may omit the chamfers 607 on the other edge of the ridge 604, instead having a straight 90 degree angle. In other embodiments, the chamfers 607 may be omitted entirely, and the edges of the ridges 604 facing both rotational directions may have straight, e.g., 90 degree angles. In certain embodiments, the ridges 604 may also be angled or pie-slice shaped, to reduce spacing near the inner diameter of the annular plate 603. The ridges 610 on the annular plate 609 of the second member 608 are arranged in substantially the same manner as described above with respect to the ridges 604 on the annular plate 603.

In use, the annular plate 603 including the ridges 604 interfaces with the annular plate 609 including the ridges 610. The ridges 604 are configured to mate with the ridges 610 such that, if chamfers 607 are present on ridges 604, they engage corresponding and complementary chamfers on ridges 610 on plate 609. If chamfers 607 are not present on the ridges 604, the 90 degree angles of ridges 604 are configured to engage corresponding 90 degree angles on ridges 610. Upon rotation of the driver 302, the first member 606 is configured to rotate. If the ridges 604 are not already in contact with the ridges 610, and the ridges 604 travel across a valley between ridges 610 before engaging ridges 610, a lag may occur between the initiation of rotation of the first member 606, and the initiation of rotation of second member 608. Nonetheless, once engagement commences between the ridges 604 and 610, rotation of the first member 606 drives rotation of the second member 608 due to force on the engaging and corresponding surfaces of the ridges 604, 610. As a result, the second member 608 rotates at the same speed as the driver 302 and the first member 606.

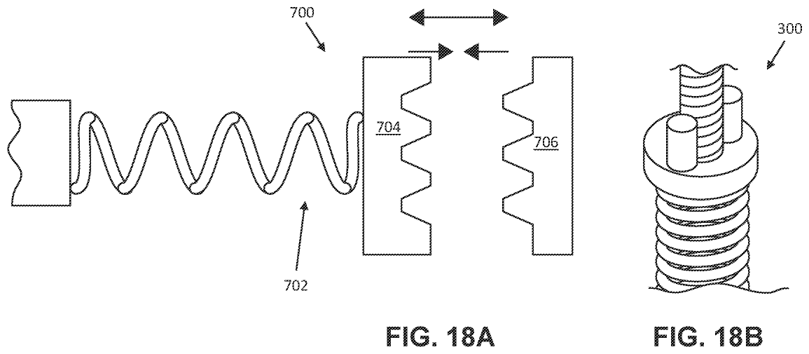

FIG. 18A illustrates an embodiment of an interface 300 in which a spring loaded/magnetic clutch 700 is used as both the driving mechanism and a parking break. Selective disconnection with the clutch may be permitted. For example, an external magnet (not shown) may be used to engage or disengage the clutch. Any one or more of the interfaces 300 described elsewhere herein can be modified with such a feature. For instance, the mechanisms can be in a default connected or default disconnected state. Such default state can be achieved using a spring 702 or other feature to urge the default connected or disconnected state. The force of the spring 702 can be selected to be overcome by external actuation. For instance, the presence or absence of an external magnet can cause physical movement of one or both sides 704, 706 of the connection (or a bridging component between the two connections) to achieve or break the connected state. Such a feature can beneficially resist unwanted torque transfer across the connection. One or both of the components 704, 706 can be magnets themselves or configured to be affected by a magnetic field induced by an external component. In some instances, the implant can include an actuator to achieve or break the connection.

Each of the foregoing torque transfer interfaces may be used to transfer torque in a distraction and compression system, including, e.g., intramedullary, extramedullary, and spinal distraction and compression systems and implants. They may also be used to transfer torque in combination with any driver of rotational motion, including a permanent magnet driven by an externally generated magnetic field, a motor, or other driver. They may also be used in combination with any other features of such implants as is understood in the art.

As used herein, the terms “first,” “second,” and the like, do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the stated value and has the meaning dictated by the context (e.g., includes the degree of error associated with measurement of the particular quantity). The suffix “(s)” as used herein is intended to include both the singular and the plural of the term that it modifies, thereby including one or more of that term (e.g., the metal(s) includes one or more metals). Ranges disclosed herein are inclusive and independently combinable (e.g., ranges of “up to about 25 mm, or, more specifically, about 5 mm to about 20 mm,” is inclusive of the endpoints and all intermediate values of the ranges of “about 5 mm to about 25 mm,” etc.).

While various embodiments are described herein, it will be appreciated from the specification that various combinations of elements, variations or improvements therein may be made by those skilled in the art, and are within the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An implant comprising:
a biocompatible housing;
a driver;
a receiver; and
an interface configured to transfer torque from the driver to the receiver, the interface comprising:
a first magnetic member rotationally fixed to the driver, wherein the first magnetic member comprises a dipole magnet, and a rotation of the driver is configured to cause a rotation of the first magnetic member; and
a second magnetic member rotationally fixed to the receiver, wherein a rotation of the second magnetic member is configured to cause a rotation of the receiver,
wherein the first magnetic member is configured to attract the second magnetic member, and the second magnetic member is configured to rotate in response to a rotation of the first magnetic member.

2. The implant of claim 1, wherein the second magnetic member is configured to rotate more slowly than the first magnetic member.

3. The implant of claim 2, wherein the second magnetic member comprises a dipole magnet.

4. The implant of claim 3, wherein the first magnetic member is magnetically coupled with the second magnetic member.

5. The implant of claim 3, wherein the first magnetic member comprises a disc-shaped dipole magnet, and the second magnetic member comprises a disc-shaped dipole magnet.

6. The implant of claim 5, further comprising a component disposed between the first magnetic member and the second magnetic member, the component including a thrust bearing or a friction reducing component.

7. The implant of claim 3, wherein the first magnetic member comprises a cross sectional shape configured to provide a complementary fit with a cross sectional shape of the second magnetic member.

8. The implant of claim 7, wherein the second magnetic member is disposed at least partly within the first magnetic member in a male to female arrangement, and wherein the first magnetic member and the second magnetic member are substantially axially aligned.

9. The implant of claim 8, wherein the first magnetic member further comprises a barrel shape, and the second magnetic member further comprises a solid cylindrical shape,
wherein the first magnetic member and the second magnetic member are substantially concentrically arranged.

10. The implant of claim 1, wherein the second magnetic member comprises a magnetic material.

11. The implant of claim 10, wherein the first magnetic member comprises a cross sectional shape configured to provide a complementary fit with a cross sectional shape of the second magnetic member.

12. The implant of claim 11, wherein the second magnetic member is disposed at least partly within the first magnetic member in a male to female arrangement, and wherein the first magnetic member and the second magnetic member are substantially axially aligned.

13. The implant of claim 11, wherein the first magnetic member further comprises a first engagement surface, and the second magnetic member comprises a second engagement surface,
wherein the second magnetic member is configured to rotate in response to force exerted by the first engagement surface on the second engagement surface.

14. The implant of claim 13, wherein the second magnetic member comprises a cross sectional geometry selected from: a semicircle, an irregular shape having at least one arc and one flat side, an irregular shape having at least one arc and two flat sides, an irregular shape having at least one arc and three flat sides, an irregular shape having at least one arc and four flat sides, a slotted circle, a cross or Phillips head, a hexalobular shape, and a keyed shape.

15. An implant comprising:
a biocompatible housing;
a driver;
a receiver; and
an interface configured to transfer torque from the driver to the receiver, the interface comprising:
a receptacle disposed on an end of the driver, and configured to accommodate an end portion of the receiver therein;
a first receptacle groove and a second receptacle groove, each disposed on an annular inner side wall of the receptacle;
a first receiver groove and a second receiver groove, each disposed on a radially outer surface of the end portion of the receiver,
wherein, when the end portion of the receiver is disposed within the receptacle, the first receptacle groove and the first receiver groove mate to form a first annular space, and the second receptacle groove and the second receiver groove mate to form a second annular space;
a first toroidal spring disposed in the first annular space, canted in a first direction;
a second toroidal spring disposed in the second annular space, canted in a second direction, opposite the first direction.

16. The implant of claim 15, wherein each of the first toroidal spring and the second toroidal spring comprise a coil spring having a first end thereof welded to a second end thereof.

17. The implant of claim 15, wherein the first toroidal spring and the second toroidal spring have a same diameter.

18. The implant of claim 15, wherein the first toroidal spring has a first diameter, and the second toroidal spring has a second diameter that is different than the first diameter.

19. The implant of claim 18, wherein the first end of the receiver comprises a stepped diameter shaft, and
wherein the first annular space having the first diameter is disposed at a first step, and the second annular space having the second diameter is disposed at a second step.

20. The implant of claim 18, wherein the end portion of the receiver further comprises a shaft and a collar disposed thereon,
wherein one of the first annular space or the second annular space is disposed on the collar, and the other of the first annular space or the second annular space is disposed on the shaft.

21. An implant comprising:
a biocompatible housing;
a driver;
a receiver; and
an interface configured to transfer torque from the driver to the receiver, the interface comprising:
a first member rotationally fixed to the driver, wherein a rotation of the driver is configured to cause a rotation of the first member; and
a second member rotationally fixed to the receiver, wherein a rotation of the second member is configured to cause a rotation of the receiver,
wherein the second member is configured to rotate in response to a rotation of the first member.
